(19) United States
(12) Patent Application Publication
(10) Pub. No.: US 2007/0249046 A1
(43) Pub. Date: Oct. 25, 2007

(54) Apparatus and method for the static application of therapeutic ultrasound and stem cell therapy of living tissues

(76) Inventor: Donald J. Shields JR., Arcadia, CA (US)

Correspondence Address: SEED INTELLECTUAL PROPERTY LAW GROUP PLLC, 701 FIFTH AVE, SUITE 5400, SEATTLE, WA, 98104, US

(21) Appl. No.: 11/408,820

(22) Filed: Apr. 20, 2006

(51) Int. Cl. C12N 5/08; A61H 1/00

(52) U.S. Cl. 435/366; 601/2

(57) ABSTRACT

Devices and methods for treating tissue injuries and augmenting the maturation and differentiation of stem cell populations are provided. The devices comprises ultrasonic oscillators capable of variable frequency output, user-selectable based on the tissue being treated. The devices produce therapeutic ultrasound that is pulsed and has a modulated amplitude and varying waveform of variable intensity.

### FIELD OF THE INVENTION

The present invention is directed to an improved apparatus and method for in vivo treatment of diverse cell, tissue and injury types, including stem cells. More particularly, the present invention is directed to in vivo ultrasound therapy for various cells, tissues and injuries through the static placement of ultrasonic treatment elements on the injury site.

### BACKGROUND OF THE INVENTION

Humans and animals are composed of cells organized into various functional units or tissue types. These tissue types include bone, muscle, tendon, ligament and cartilage, all of which are commonly injured. Therapeutic ultrasound has long been used as one mode of treating injuries to these tissues, as well as for treating a variety of other tissues and injuries.

The thermal effects of therapeutic ultrasound treatment are well known and are widely considered the reason for the success of the treatment. However, these thermal effects can also damage the target tissue if the transducer treatment elements are not kept in constant motion. This risk requires conventional ultrasound devices to be operated by trained and knowledgeable personnel.

Research into the non-thermal effects of therapeutic ultrasound has uncovered other mechanisms of action such as cavitation and acoustic streaming which have been shown to cause the up-regulation of many cellular processes. These non-thermal effects of therapeutic ultrasound have been found to produce more efficient inflammatory reactions and improved time and quality of healing in a wide variety of tissue types.

Low-intensity therapeutic ultrasound has been shown to produce these same beneficial, non-thermal effects without creating an increased temperature in the treated tissues. Unlike higher intensity ultrasound therapy, low-intensity ultrasound treatment enables the placement of the treatment element in a static position on the patient without thermally damaging the target tissue. Accordingly, these low-intensity devices are very easy to use, thereby improving patient compliance.

The effects of therapeutic ultrasound on living tissues vary, but the greatest impact occurs on highly organized, structurally rigid tissues such as bone, tendons, ligaments, cartilage and muscle. Increased healing of these tissue types can be achieved when either high- or low-intensity therapeutic ultrasound is employed. Due to their different depths within the body, however, the different tissue types require different ultrasonic frequencies for effective treatment. In addition, tissues will respond to ultrasound in different ways depending on the chronicity of the injury to the tissue. As a result, acute and chronic injuries are treated differently. Typically, chronic injuries are treated with higher intensity ultrasound, a pulse ratio of about 1:2, and a waveform pulse duration of about 100 μs to produce good healing properties. In contrast, acute injuries are generally treated with lower intensity ultrasound, a larger pulse ratio of about 1:4 and a longer waveform pulse duration of about 200 μs to produce good healing properties.

Apparatuses and methods utilizing these scientific principles have been disclosed, including apparatuses and methods of using ultrasonic energy for in vivo therapeutic treatment of bone tissue with carrier frequencies and therapeutic ultrasound pulses. While these apparatuses often allow for the selection of certain treatment variables such as ultrasound frequency, pulse intensity and other pulse waveform characteristics, the apparatuses produce a single, fixed waveform during each treatment application. As such, these apparatuses and methods are designed to treat a single type of tissue with a single, specific and fixed ultrasonic frequency, pulse intensity, pulse ratio, pulse duration and pulse repetition rate during each therapeutic use. Because these apparatuses use such singular waveforms, the transducer treatment elements must be kept in constant motion to avoid thermal damage to the target tissue.

### SUMMARY OF THE INVENTION

The present invention is directed to methods and apparatuses for the safe application of therapeutic ultrasound via the static application of the transducer treatment element. By manipulating the waveform characteristics of the ultrasonic pulses utilized for therapy and the treatment time during which each of these pulses is applied, a controlled and safe increase in the target tissue temperature may be achieved. By repeatedly cycling the ultrasonic waveform characteristics of intensity, pulse duration, pulse ratio and pulse repetition rate between settings which produce a rise in target tissue temperature and settings which do not produce such a rise in temperature, thermal damage is avoided.

To that end, the present invention is directed to in vivo ultrasound therapy for musculoskeletal tissue and other tissues and/or cell types, including stem cells. According to the inventive therapy, ultrasonic energy is delivered to target tissue. The ultrasound therapy device generally comprises at least one ultrasound treatment element, e.g. a transducer. The ultrasound treatment element may comprise a single element, multiple elements or an array of elements, but the ultrasound therapy device is designed to treat a wide variety of tissue types and treatment areas within a human or animal body through use of a single therapy device.

In one embodiment, the therapy device produces low to high intensity, pulsed ultrasonic waveforms having variable frequency, intensity and waveform characteristics. Such a design enables effective treatment of tissues and/or cells regardless of the depth of the tissue in the body or the chronicity of the injury to the tissue.

In another embodiment of the present invention, ultrasound therapy is used to facilitate differentiation and/or maturation of stem cells. In this embodiment, the stem cells themselves may be treated with ultrasound therapy. Alternatively, the stem cells may be combined with a bioactive compound, such as a member of any of the growth factor families, including but not limited to insulin-like, tissue or bone growth factors. The bioactive compound may be added before, during or after treatment of the stem cells with the ultrasound therapy, and the addition of such a bioactive compound augments the effects of the ultrasound treatment.

An ultrasound therapy device according to one embodiment of the present invention applies ultrasound energy of varying frequency, intensity and waveform characteristics to injured cells or tissues to promote healing. The device delivers ultrasound energy non-invasively through at least one ultrasound treatment element (each of which can include a single element or an array of elements) placed on the patient's skin. The ultrasound energy is delivered from a control module to the treatment element either through a wireless radio frequency connection or through a cable connecting the treatment element to the control module. The cable may be any suitable cable for connecting the control module to the treatment element, including but not limited to such conventional cables as electrical cables, radio-frequency (RF) cables and fiber-optic cables.

Typically, the treatment elements comprise ultrasound transducers which direct sound waves to the injured tissue, thereby healing the tissue. Any conventional transducer suitable for use with ultrasound diagnostic or therapeutic equipment that produces ultrasonic waves of the desired frequencies can be used with the present invention. In one embodiment, each transducer comprises a circular disc with an outer diameter of about 1 inch and includes a treatment surface and a visible surface. The treatment surface is the surface of the transducer which contacts the patient's skin during treatment and which delivers the ultrasound waves. The visible surface is the surface opposite the treatment surface which is visible during treatment. As noted above, a single transducer, an array of transducers, or a plurality of transducers may be used.

In addition, combined treatment heads may be used. Such combined treatment heads contain multiple ultrasonic treatment elements capable of producing a plurality of treatment frequencies. All treatment elements of the combined treatment head produce the same treatment waveform, but at different frequencies, thereby allowing simultaneous treatment of tissues at multiple depths within the body.

In one embodiment, the treatment elements (transducers) are contained in an applicator. To use the devices of the present invention, a coupling gel is first placed on the skin of the patient, and the treatment elements are placed over the coupling gel directly over the intended treatment site. Any conventional coupling gel suitable for use with ultrasound therapeutic or diagnostic applications can be used with the present invention.

The waveform characteristics are generated by any suitable means, such as by a radio frequency oscillator and a pulse generator, both of which are user-controlled to produce a variety of desired ultrasonic frequencies and waveform pulses. The ability to control the frequency and waveform pulse of the ultrasound energy directs the effectiveness of the ultrasound treatment. The ability to divide the total treatment into a combination of unique and variable treatment segments further drives the safety and effectiveness of the ultrasound treatment delivered by the inventive devices. The treatment segments are produced individually for the desired length of time and the device cycles through the various treatment segments until all segments have been used and the total treatment time has elapsed.

As noted above, the transducers can be controlled via a cable connecting the transducers to the control module or wirelessly via radio communication with the control module. In either embodiment, the control module can control a plurality of separate transducers. However, the number of transducers connectable to the control module by cables is limited. Accordingly, in the wireless embodiment, the control module can control more transducers.

The devices according to the present invention can be used to heal a variety of different cells and tissues, including bone, cartilage, tendons and ligaments, and muscles. The piezoelectric nature of bone enables conversion of the mechanical ultrasonic energy to an electric current in the bone. The electric current in the bone directly promotes bone growth in the target bone tissue, thereby promoting the healing of the injured bone.

Unlike the conversion of ultrasonic energy to an electric current that occurs with ultrasound therapy, non-ultrasonic bone stimulators either generate the current directly or induce the current using an external electromagnetic field. Non-ultrasonic, direct current bone growth stimulators apply current continuously, and at least three months of treatment is usually required to achieve a therapeutic response. In addition, non-ultrasonic, electromagnetic treatments usually require 12-16 hours of treatment per day, and the total treatment time is normally even longer than the treatment time needed for direct current growth stimulators. In contrast, ultrasonic treatment according to one embodiment of the present invention involves application of ultrasound energy for about 20 to about 45 minutes per day, for a total treatment time of about two months or less. Using the ultrasonic treatment of the present invention not only heals the bone defect much faster, but also requires considerably less treatment time.

The ultrasound therapy devices of the present invention effect healing through both non-thermal effects such as acoustic streaming and cavitation, which cause the up-regulation of many cellular processes, leading to more efficient inflammatory processes, as well as safe and beneficial thermal effects. These processes have been shown to improve the quality of healing and to decrease the time needed to heal.

As noted above, the devices of the present invention can be used to heal a variety of different cells and tissues. To treat such a variety of tissue types, certain operating characteristics are variable and selectable by the user based on the tissue being treated and the type of injury to the tissue. In one embodiment, the device outputs a frequency of ultrasound energy ranging from about 50 kHZ to about 3 MHz. In addition, the devices apply ultrasound energy in pulses, and the duration of each energy pulse ranges from about 10 to about 2,500 microseconds (μs). The intensity of each pulse ranges from bout 20 mW/cm² to bout 3 W/cm². The pulse repetition rate ranges from about 50 to about 10,000 Hz. The pulse ratio ranges from about 1:1 to about 1:8. These waveform characteristics are variable and user-selectable based on the tissue being treated and the type of injury to the tissue.

The ultrasound intensity (power) is also variable, but must be maintained below a safety threshold level in order to prevent damage to the target and adjacent tissues. However, it is power density over time that is important, rather than the absolute total power level (i.e. the area of the transducer multiplied by the power per unit area). Intensity can be varied within a single pulse to output a multi-variant waveform. Alternatively, intensity can be varied within a series of pulses, where each pulse has a fixed waveform, but the waveform varies from pulse to pulse. The intensity can also be varied both within a single pulse and within a series of pulses such that one pulse in a series has a fixed waveform and another pulse in the same series has a multi-variant waveform.

A method of treating tissue according to another embodiment of the present invention includes applying ultrasound energy to the target tissue with the inventive device for about 5 to about 60 minutes per day. In one embodiment, each treatment session ranges from about 20 to about 60 minutes and a patient undergoes from 1 to 3 sessions per day. In one embodiment, the tissue is treated for about 30 to about 45 minutes per day. Daily treatment is continued until the injured tissue is healed. The required frequency of the ultrasound depends on the depth of the target tissue within the body, and is therefore selected based on the type of tissue to be treated. The intensity and waveform characteristics utilized during therapy depend upon both the type of injury to be healed and the tissue being treated. Chronicity of the injury is one of the main factors affecting the selection of intensity and waveform settings. Acute lesions are more sensitive to treatment than chronic lesions and therefore require less intensity.

The methods of treatment according to the present invention enable selection and variation of treatment parameters such as frequency, intensity and waveform characteristics based on the patient, the tissue being treated and the type of injury to that tissue. The devices and methods of the present invention can be used even if the area of the body to be treated is enclosed in a cast. To treat such an area, a window is cut in the cast directly above the injury site.

In another embodiment of the present invention, therapeutic ultrasound is used in conjunction with stem cell therapy. The stem cell therapy may be used either by itself or in conjunction with non-biophysical methods of tissue repair and regeneration, such as bioactive compounds. Non-biophysical methods of tissue repair and regeneration include use of bioactive compounds (e.g. growth factors) to stimulate tissue repair and growth. These treatment modalities may be used in any combination. For example, ultrasound may be used by itself, in conjunction with stem cell therapy or in conjunction with stem cell therapy and non-biophysical methods. These treatment modalities may be delivered in any combination or temporal relationship.

When using ultrasound in conjunction with stem cells or with stem cells and bioactive compounds, a synergistic effect is achieved and the injured target tissue is healed more effectively and in less time. In particular, when ultrasound is combined with stem cells and/or bioactive compounds, the total days of treatment are shortened, thereby effecting successful tissue healing, regeneration and/or repair more efficiently.

In treating bone fractures, healing is improved and healing time decreased when bioactive compounds are used in conjunction with therapeutic ultrasound, as compared with the use of ultrasound therapy alone. The differentiation and maturation of stem cell populations is also enhanced by coupling therapeutic ultrasound and bioactive compounds, as compared to the use of stem cells in the presence of bioactive compounds alone.

While the effect of therapeutic ultrasound on stem cell populations alone is not well documented, it is likely that the up-regulation of other cells undergoing ultrasound therapy affects the stem cells. When stem cells are implanted into the target tissue, environmental factors of the parent tissue, such as extra-cellular matrix compounds, affect and influence the cell lineage into which these multi-potent cells differentiate. The up-regulation of the parent cells results in greater cell lineage influence on the implanted stem cell population. However, with the application of therapeutic ultrasound, the up-regulation of the stem cells themselves will result in an increased number of cells undergoing differentiation and decreased differentiation/maturation times.

The devices and methods of the present invention exhibit improved tissue healing by providing interchangeable transducer treatment elements. A single transducer, a transducer array or a plurality of transducers may be provided to more effectively cover treatment areas of any size. A variable frequency ultrasonic generator or multiple fixed frequency generators are used to produce the frequency necessary to concentrate the ultrasound waveform pulse at the target tissue depth. Intensity of the ultrasound signal is also varied, as are the pulse ratio, pulse duration and pulse repetition rate which comprise the waveform characteristics. These characteristics are varied by the user based on the tissue type and the chronicity of the injury to be treated. For example, chronic injuries are less sensitive to the ultrasound therapy, therefore requiring more intense therapy. Variations in ultrasound frequency, intensity and waveform characteristics may also be predetermined and programmed into the device by the manufacturer. These predetermined variations are preset by the manufacture based on tissue and injury classifications and are accessible from the control module.

### BRIEF DESCRIPTION OF THE DRAWINGS

These and other features and advantages of the present invention will be better understood by reference to the following detailed description when considered in conjunction with the accompanying drawings in which:

FIG. 1 is a schematic of a therapy device according to one embodiment of the present invention;

FIG. 1a is a schematic of a therapy device according to another embodiment of the present invention;

FIG. 2 is a diagram of the connectivity of various components of the device of FIG. 1;

FIG. 3a is a schematic of a single transducer according to one embodiment of the present invention;

FIG. 3b is a schematic of a transducer array according to another embodiment of the present invention;

FIG. 4a is a schematic of an exemplary fixed waveform output by a therapy device according to one embodiment of the present invention;

FIG. 4b is a schematic of an exemplary multi-variant waveform output by a therapy device according to another embodiment of the present invention;

FIG. 5 is a schematic of a therapy device according to another embodiment of the present invention;

FIG. 6a is a diagram of the connectivity of various components of the control module of the device of FIG. 5;

FIG. 6b is a diagram of the connectivity of various components of a transducer of the device of the FIG. 5;

FIG. 7 is a schematic of a therapy device according to yet another embodiment of the present invention; and

FIG. 8 is a diagram of the connectivity of various components of the device of FIG. 7.

### DETAILED DESCRIPTION OF THE INVENTION

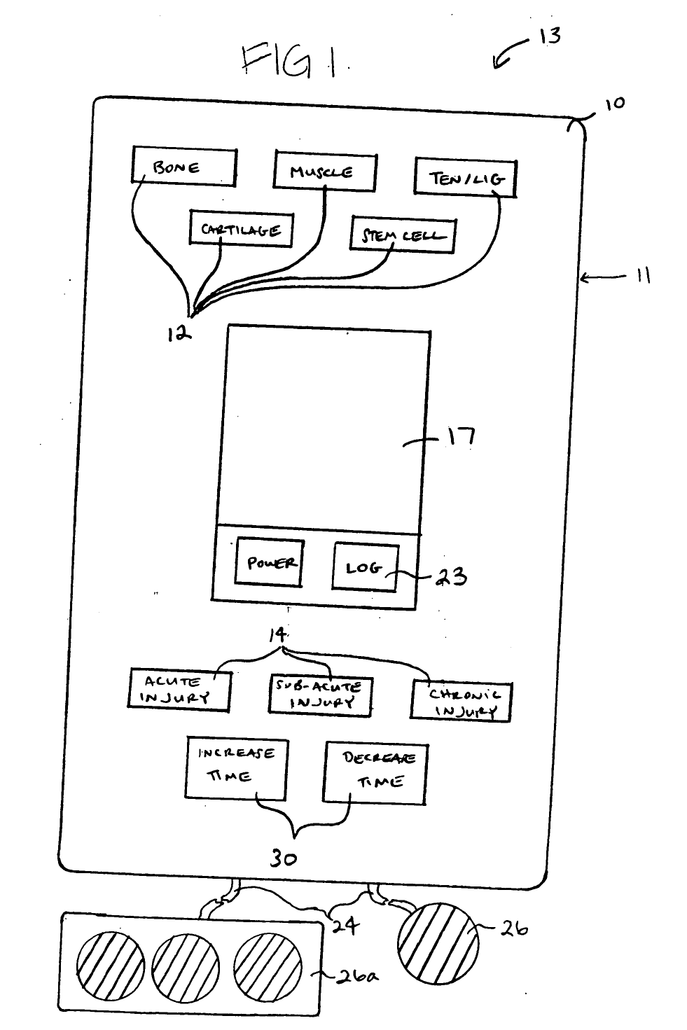

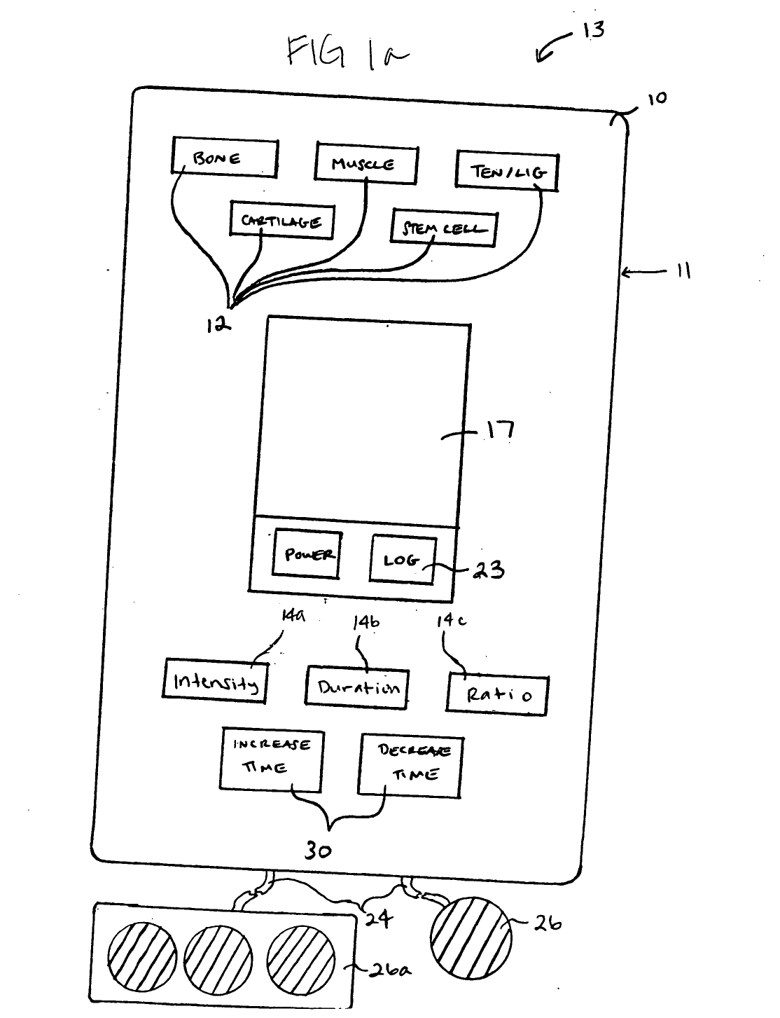

In one embodiment of the present invention, as shown in FIGS. 1 and 2, a device 10 for applying ultrasound energy to target tissue comprises a control module 13 having a housing 11 with at least a first selector 12 for selecting the type of tissue to be treated. The first selector 12 can have any construction suitable for effecting selection. For example, each selector may comprise a button which effects selection upon depression. Alternatively, the housing 11 may include a touch-sensitive screen having areas designated for tissue selection and which effects selection upon touching the desired area of the screen. The first selector may alternatively comprise a single selector capable of scrolling, rotating or otherwise effecting selection. In one exemplary embodiment, the first selectors 12 are adapted to enable selection of tissue. Nonlimiting examples of selectable tissue include bone, muscle, cartilage, tendons and/or ligaments, and stem cells. Selection of tissue with the first selectors 12 determines the ultrasonic frequency of the pulses generated by the device 10.

In addition, the device 10 can include at least one second selector 14 for selecting the type of injury to be treated. The second selectors 14 in this embodiment are pre-programmed to deliver waveform characteristics based on the type of injury selected. Depending on the injury selected, the second selectors 14 deliver waveforms having varying characteristics, such as the length of each waveform segment (described in more detail below), and pulse intensity, pulse duration, pulse ratio and pulse repetition rate of each segment. The second selectors 14 also determine the order in which the device cycles through each treatment segment.

The second selector 14 can have any construction suitable for effecting selection. For example, each selector may comprise a button which effects selection upon depression. Alternatively, the housing may include a touch-sensitive screen having areas designated for injury selection and which effects selection upon touching the desired area of the screen. The second selector may alternatively comprise a single selector capable of scrolling, rotating or otherwise effecting selection. In one exemplary embodiment, the at least one second selector 14 includes three selectors, one each for chronic injuries, sub-acute injuries, and acute injuries.

Alternatively, as shown in FIG. 1a, the second selectors 14 comprise separate selectors 14a, 14b and 14c for controlling the waveform characteristics separately. In this embodiment, the pulse intensity is controlled by second selector 14a, the pulse duration is controlled by second selector 14b and the pulse ratio is selected by second selector 14c. The combination of pulse duration and pulse ratio generates the pulse repetition rate. As such, the pulse repetition rate is automatically generated by the selection of the pulse duration and pulse ratio. Because this fine tuning of treatment variables requires extensive knowledge of therapeutic principles and medical practices, this embodiment is designed for use by qualified individuals.

In this embodiment, the ultrasonic frequency is selected by tissue type through the first selectors 12. Pulse intensity is selected next because the selection of pulse intensity affects the available parameters for the remaining waveform characteristics. The second selectors can be pre-programmed to prevent selection of combinations of waveform characteristics and treatment segment times that may lead to thermal or other tissue damage. Once pulse intensity is selected, the pulse duration is then selected. The pulse ratio is then selected, and finally, the treatment time is set. After setting the first waveform, the user may set another waveform, or simply begin treatment with a single waveform.

The first and second selectors 12 and 14, respectively, are used to select the treatment parameters. Based on the selections made with the first and second selectors, a waveform is generated for treating the selected tissue. The waveform generated comprises a plurality of waveform segments, each segment having at least one characteristic different from the previous or subsequent waveform segment. The different waveform segments may differ in one or more characteristics such as pulse ratio, pulse intensity, pulse duration or pulse repetition rate. Upon selection with the first and second selectors, the device will begin cycling through the selected waveform segments. Each waveform segment is activated for a certain period of time before the device cycles to the next segment. The device continues to cycle through the waveform segments until all the segments have been used and the total treatment time elapses.

In one embodiment, the second selectors 14 are eliminated and the first selectors 12 are pre-programmed by the manufacturer to deliver a specific waveform based on the tissue selected. The pre-programmed treatment may include variations in waveform characteristics such as the length of each treatment segment and the cycling order of the treatment segments.

Each of the first selectors 12 and second selectors 14 communicate with a waveform generator 15. The waveform generator 15 includes a radio frequency (RF) oscillator 16 and a pulse generator 18. In one embodiment, the waveform generator 15 includes a single variable frequency RF oscillator 16 programmed to deliver different frequencies based on the type of tissue selected. In an alternative embodiment, the waveform generator 15 includes a plurality of RF oscillators, one for each type of tissue. In such an embodiment, each of the RF oscillators is pre-programmed to transmit a specific frequency based on the tissue type with which the RF oscillator is associated.

Although the RF oscillator generates a signal based on the type of tissue selected, the RF oscillator 16 generally generates a signal with a frequency ranging from about 20 kHz to about 3 MHz. The frequency needed to reach each tissue type is generally known in the art.

The pulse generator 18 controls the pulse intensity, duration, ratio and repetition rate, each of which is determined based on the type of injury selected by the second selectors 14 and the tissue selected by the first selectors 12. Although dependent upon the injury selected, the pulse generator 18 generally generates a pulse having a pulse duration ranging from about 10 to about 2,500 μs, a pulse repetition rate (frequency) ranging from about 50 to about 10,000 Hz and a pulse intensity ranging from about 20 mW/cm² to about 3 W/cm². In one exemplary embodiment, the pulse frequency is about 1,000 Hz and the pulse duration ranges from about 100 to about 200 μs.

The pulse generator generates pulsed periods and non-pulsed (or inactive) periods. The pulse ratio refers to the ratio of time that the pulse generator is active (generating pulsed periods) to the time that the pulse generator is inactive (generating non-pulsed periods). According to one embodiment of the present invention, the pulse generator generates pulsed and non-pulsed periods in a pulse ratio dependent on the type of injury selected. In general, however, the pulse ratio ranges from about 1:1 to about 1:8. In one exemplary embodiment, for chronic injuries, the pulse ratio ranges from about 1:1 to about 1:2, and for acute injuries, the pulse ratio ranges from about 1:3 to about 1:4.

The pulse generator 18 varies the intensity of the pulses depending on the type of tissue and injury selected. For example, for chronic injuries, the pulse generator 18 outputs a pulse having a greater intensity than the pulse output for acute injuries. Although dependent on the type of injury and tissue selected, the pulse generator 18 generally varies the intensity of the pulse within a range of between 20 mW/cm² and 3 W/cm².

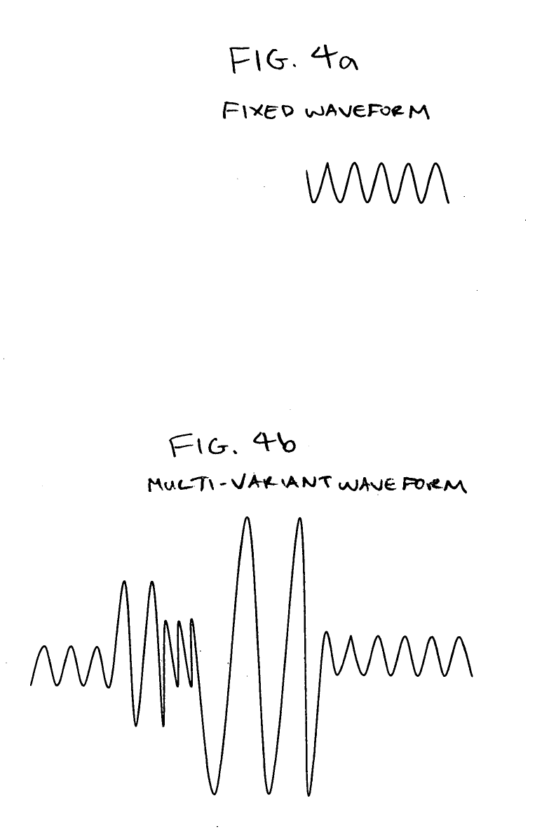

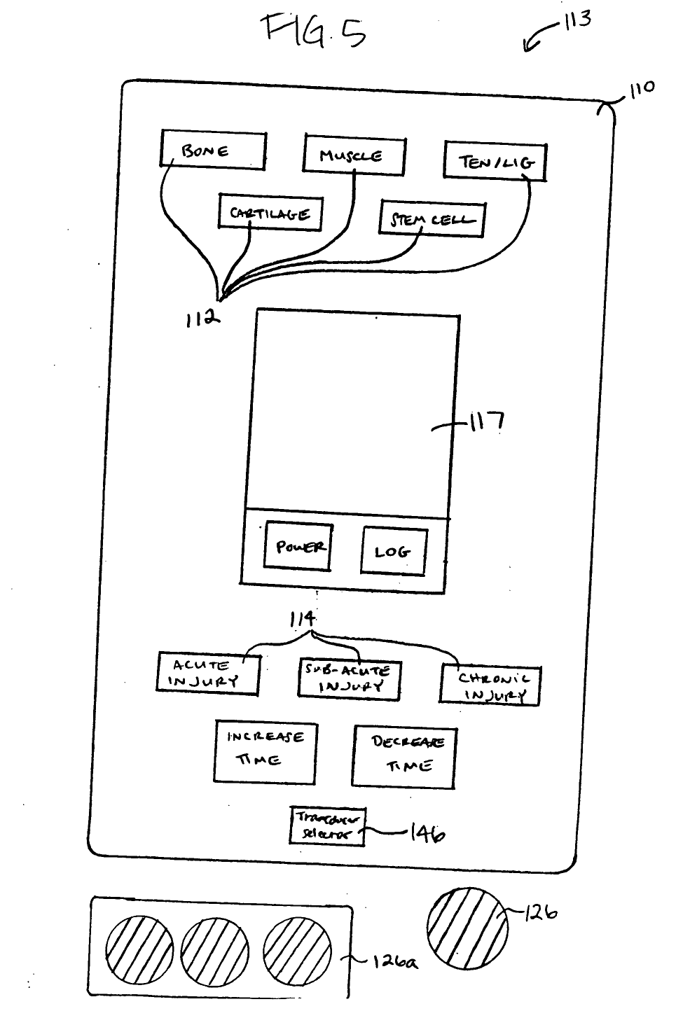

The pulse generator 18 can vary the intensity of a single pulse or a series of pulses throughout a single treatment session. For example, the pulse generator 18 can vary the intensity of a single pulse in a series of pulses delivered in a single treatment session such that each pulse output by the pulse generator has a multi-variant waveform, as shown in FIG. 4b. Alternatively, the pulse generator can vary the intensity of the series of pulses, such that each pulse in the series of pulses delivered in a single treatment session has a fixed waveform (as shown in FIG. 4a). In this embodiment, the waveform of each pulse may differ. In another embodiment, the pulse generator 18 varies the intensity of each pulse and the intensity of the series of pulses delivered in a single treatment session. In this embodiment, each pulse may have either a multi-variant waveform or a fixed waveform. For example, one pulse in the series may have a fixed waveform and another pulse in the series may have a multi-variant waveform.

The waveform generator 15 is electrically connected to a driver 20 which modulates the output of the RF oscillator 16 with the signal generated by the pulse generator 18 to generate a single signal. In one exemplary embodiment, the driver 20 generates a signal in the form of a sine wave having a frequency ranging from about 50 kHz to about 3 MHz during pulsed periods. The driver 20 also amplifies the resulting signal in order to deliver power having a maximum intensity for safe and effective ultrasonic therapy. In one exemplary embodiment, the power has a maximum intensity of about 3 W/cm². In another exemplary embodiment, the driver 20 delivers power having an intensity ranging from about 30 to about 500 mW/cm².

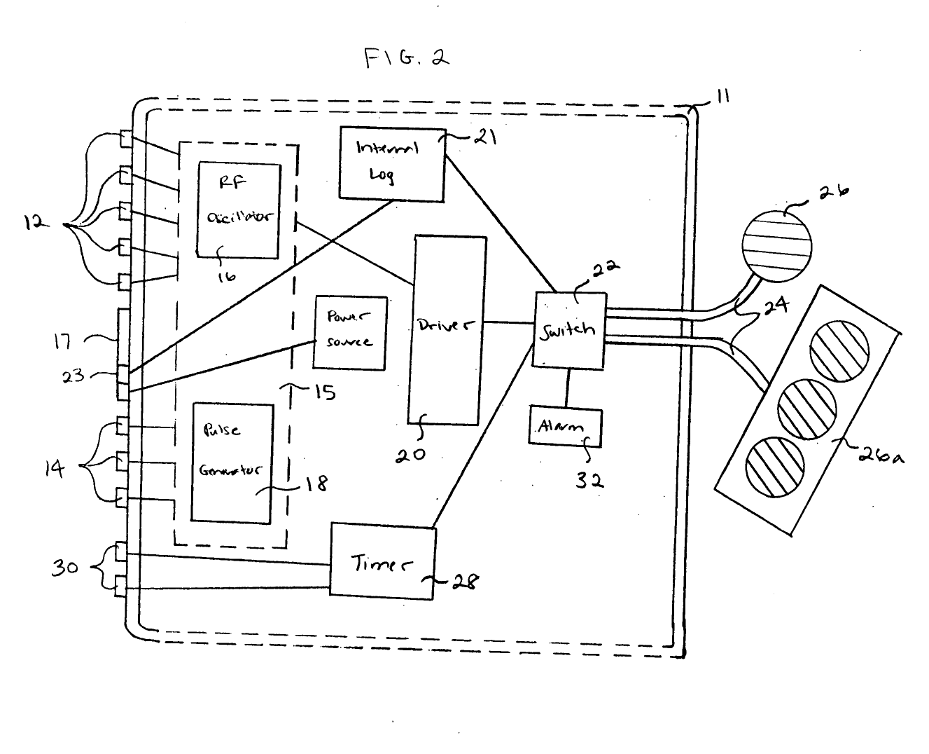

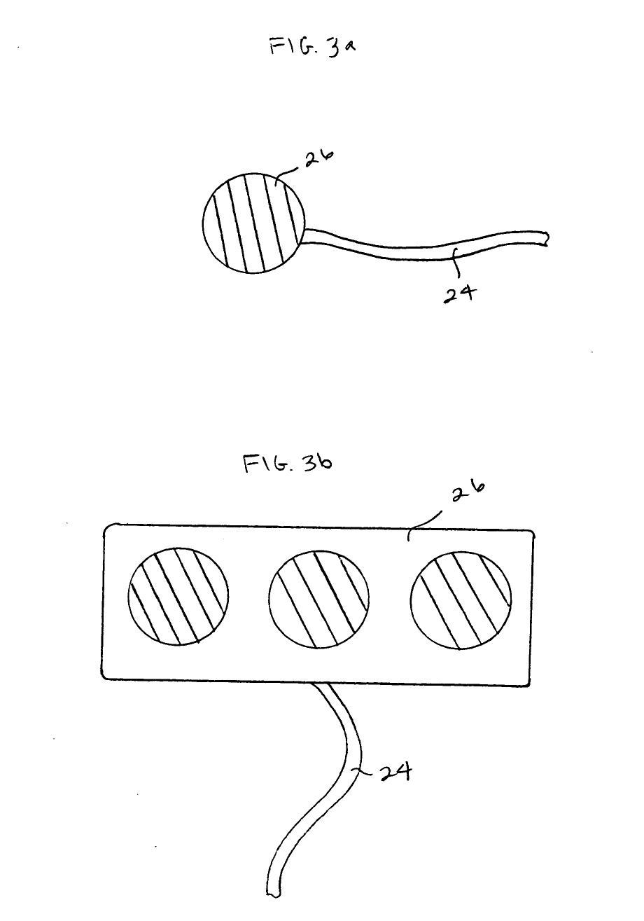

The driver 20 communicates with a switch 22. The switch 22 communicates with a transducer cable 24, which communicates with a transducer 26 (shown in FIG. 3a). In another exemplary embodiment, multiple cables 24 communicate with one or more transducers 26 and/or transducer arrays 26a (shown in FIG. 3b). In use, the switch 22 receives the signal from the driver 20 and transmits it to the cable 24 which delivers the signal to the transducer 26.

The device 10 can further include a timer 28 for timing the treatment. As shown, at least one third selector 30 is electrically connected to the timer 28 for setting the treatment time. The third selector 30 can have any construction suitable for setting the treatment time. For example, each selector may comprise a button which alters the treatment time upon depression. Alternatively, the housing may include a touch-sensitive screen having areas designated for time selection and which alters the treatment time upon touching the desired area of the screen. The third selector may alternatively comprise a single selector capable of scrolling, rotating or otherwise altering the treatment time. In one exemplary embodiment, the treatment time varies from about 5 to about 60 minutes per session and from 1 to 3 sessions per day. In another exemplary embodiment, however, the treatment time ranges from about 30 to about 45 minutes per session. Daily treatments continue until the injured tissue is healed.

The timer 28 is electrically connected to the switch 22. Upon setting the timer 28 and starting the treatment, the switch 22 transmits the signal from the driver 20 to the cable 24. When the treatment time has elapsed, the switch 22 ceases to deliver the signal from the driver 20 to the cable 24, thereby ending the treatment. In one embodiment, the timer is pre-programmed with treatment times based on selected tissue and injury types. However, the timer may also include an override feature allowing the user to set treatment times outside the preset parameters.

In addition, the device 10 can include an alarm 32 electrically connected to the switch 22 for alerting the user to the expiration of the treatment time. In this embodiment, when the treatment time set with the timer lapses, the switch 22 ceases to deliver the signal from the driver 20 to the cable 24, and instead energizes the alarm 32, which alerts the user to the completion of the treatment. The alarm 32 may alert the user by any suitable means, such as an audible alarm that rings or otherwise makes a noise indicating the completion of the treatment. Alternatively, the alarm may alert the user by visual means, such as flashing lights or the like.

The switch 22 may also comprise an interrupt feature for pausing treatment when a loss of contact between the transducer and the treatment area occurs or when the transducer is otherwise not functioning properly. When such an event takes place, the switch will cease delivering the signal from the driver 20 to the transducer 26, and will energize the alarm 32 to alert the user to the malfunction.

The device 10 may further include at least one display screen 17 and an internal log 21 for documenting usage of the device 10 and treatment specifics. In addition, the device 10 may include means for entering patient data. For example, the control module 13 may include a keyboard or the like for entering patient data, such as the patient's name, age, weight, injury complained of, etc. The display screen 17 can be any suitable screen for displaying the desired information, for example a liquid crystal display screen. Also, at least two display screens 17 can be provided, one for displaying information related to treatment specifics, and one displaying elapsed time during treatment.

The internal log 21 can comprise any suitable mechanism, such as a microprocessor or the like, and can be accessed by a log selector 23 located on the device 10. When accessed, the log information will appear on the at least one display screen 17. The log may store information such as the number of treatments performed, the length of each treatment, the date and time each treatment was performed, the types of tissues and/or injuries treated, etc.

The internal log 21 is electrically connected to the switch 22 and the timer 28. In use, the switch 22 delivers information to the internal log 21 which then stores the received information. Also, the log 21 stores the timing information received from the timer 28. As noted above, this stored information can later be accessed via the keyboard or any other suitable means such as a touch-screen menu or through sequential depressions of the log button, and the information is displayed on the display screen 17 for analysis by the user.

As noted above, the transducer cable 24 is electrically connected to at least one transducer 26. The transducer cable 24 may be connected to the transducer electrically or by any other suitable means such as RF or fiber-optics. The transducer 26 may be a single transducer 26 (as shown in FIG. 3a) or an array of transducers 26 (as shown in FIG. 3b). In addition, multiple transducers 26 or transducer arrays 26a may be connected through multiple cables 24 to a single device 10. Transducer arrays and multiple transducers are used when the treatment area is relatively large.

In one embodiment, the transducer is a combined frequency output transducer having multiple fixed or variable frequency transducer elements. These transducers are used to provide simultaneous therapy to various depths of thick target tissues such as inflamed joint capsules or muscle tissue.

In one exemplary embodiment, the transducers can be any conventional transducer, such as those made of piezoelectric materials. Each transducer comprises a generally round disc approximately 1 inch in diameter and includes a treatment surface and a visible surface. Although described and illustrated as a generally round disc, it is understood that the transducer can have any other suitable geometric shape.

Each transducer may also comprise at least one temperature sensor 54 for sensing the temperature of the patient's skin during treatment. The temperature sensor 54 is electrically connected to the switch 22, which is configured to cease delivery of waveform information to the transducer if the temperature of the patient's skin reaches a safety, threshold level. The transducer may further comprise means attached to the visible surface of the transducer to alert the user to a malfunction. For example, the transducer may include at least one light emitting diode (LED) 58 on the visible surface of the transducer which lights up or flashes when a malfunction occurs. Alternatively, the LED associated with the temperature sensor can be placed on the control module or on both the control module and the transducer treatment element. Instead of, or in addition to the LED, the transducer may include an audible alarm that alerts the user to the malfunction.

The device 10 may be powered by any suitable means. In one embodiment, for example, the device 10 includes an internal battery pack sufficient to power all elements of the device 10. Alternatively, the device 10 may be powered by plugging it into an electrical socket.

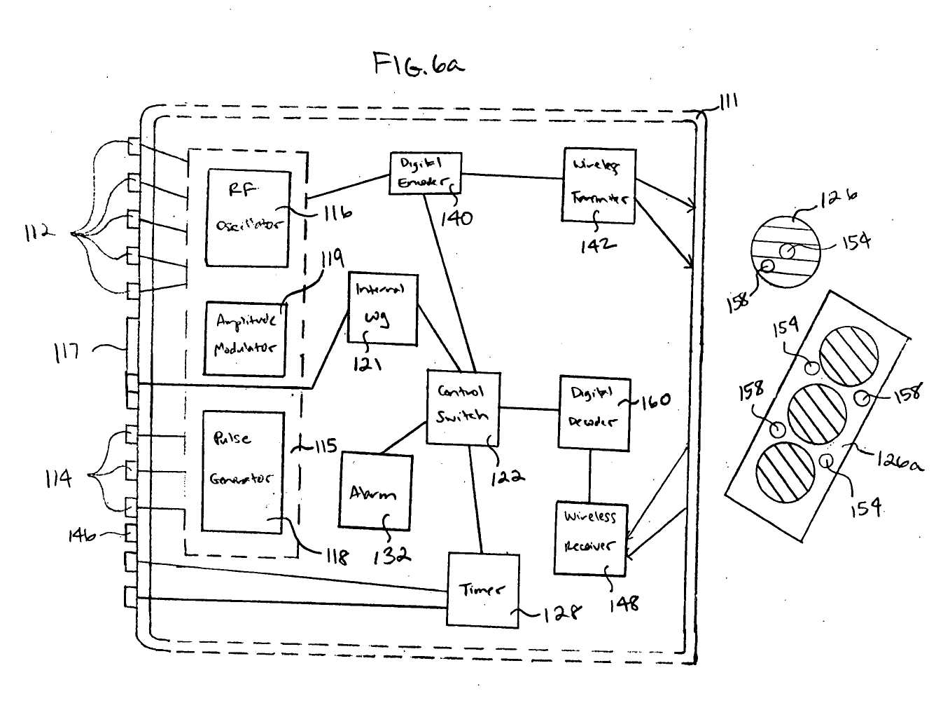

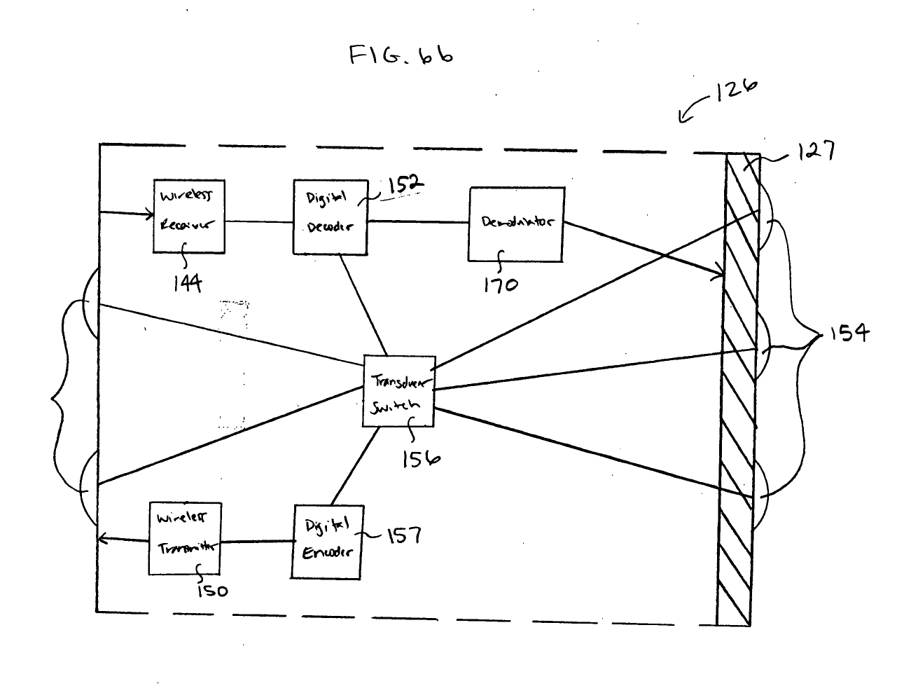

In another embodiment of the present invention, as shown in FIGS. 5, 6a and 6b, the transducers or transducer arrays are wirelessly coupled to the control module which communicates with the transducers via radio communication. Although the wireless connection is described as radio communication, it is understood that any suitable wireless connection can be used, including but not limited to optical connections, ultraviolet connections, Bluetooth® or other internet connections. In this embodiment, the device 110 comprises a control module 113 and at least one wireless transducer 126 or transducer array 126a. The control module 113 and each of the transducers 126 or arrays 126a are powered by internal batteries, rendering the device 110 completely wireless and allowing maximum portability. Alternatively, the control module may remain stationary in a fixed location such that the control module can be powered by plugging it into an electrical socket.

The device 110 comprises a control module 113 having a housing 111 with at least one first selector 112 for selecting the type of tissue to be treated. Like the first selectors 12, the first selectors 112 can have any construction suitable for effecting selection. For example, each selector may comprise a button which effects selection upon depression. Alternatively, the housing 111 may include a touch-sensitive screen having areas designated for tissue selection and which effects selection upon touching the desired area of the screen. The first selector may alternatively comprise a single selector capable of scrolling, rotating or otherwise effecting selection. The first selectors 112 are adapted to enable selection of tissue from bone, muscle, cartilage, tendons and/or ligaments, and stem cells.

In addition, the device 110 can include at least one second selector 114 for selecting the type of injury to be treated. Like the second selectors 14, the second selectors 114 can have any construction suitable for effecting selection. For example, each selector may comprise a button which effects selection upon depression. Alternatively, the housing 111 may include a touch-sensitive screen having areas designated for injury selection and which effects selection upon touching the desired area of the screen. The second selector may alternatively comprise a single selector capable of scrolling, rotating or otherwise effecting selection. In one exemplary embodiment, the at least one second selector 114 includes three selectors, one each for chronic injuries, sub-acute injuries, and acute injuries.

Each of the first selectors 112 and second selectors 114 communicate with a waveform generator 115. The waveform generator 115 is similar to the waveform generator 15 and includes a radio frequency (RF) oscillator 116 and a pulse generator 118. However, the waveform generator 115 according to this embodiment also includes an amplitude modulator 119 for modifying the waveform into a form transmittable by radio, as is generally known. The RF oscillator 116 and pulse generator 118 operate in the same fashion as described above with respect to the RF oscillator 16 and pulse generator 18. Also, like the waveform generator 15, the waveform generator 115 can include a single variable frequency RF oscillator programmed to deliver different frequencies based on the type of tissue selected. Alternatively, the waveform generator 115 can include a plurality of RF oscillators, one for each type of tissue. In such an embodiment, each of the RF oscillators is pre-programmed to transmit a specific frequency based on the tissue type with which the RF oscillator is associated.

As shown in FIGS. 6a and 6b, the waveform generator 115 communicates with a first digital encoder 140 which converts the signal received from the waveform generator 115 to a digital signal and delivers the digitally encoded signal to a first wireless transmitter 142. The first wireless transmitter 142 in the control module 113 is in radio communication with a first wireless receiver 144 in each transducer 126 or transducer array 126a. Any construction of the control module 113 and wireless transducers 126 or arrays 126a suitable for effecting radio communication can be used in this embodiment. In addition, any suitable means for radio communication between the control module 113 and the wireless transducers 126 or arrays 126a may be used.

In one exemplary embodiment, the first wireless transmitter 142 can deliver a single radio signal receivable by each of the first wireless receivers 144 in the transducers 126 or transducer arrays 126a, such that each transducer or array will output the same waveform. Alternatively, the first wireless transmitter 142 can deliver a plurality of radio signals, and the first wireless receivers 144 can be configured to receive a single signal unique to each transducer 126 or transducer array 126a such that each transducer or array will output a distinct waveform. In this embodiment, the control module 113 can include at least one fourth selector 146 for selecting which of the transducers 126 or transducer arrays 126a to which to send each signal. This construction allows the physician or user to control a plurality of transducers or transducer arrays from a remote location, and enables treatment of more than one patient at a time.

As noted above, each transducer 126 or array 126a comprises a first wireless receiver 144 for receiving radio signals from the first wireless transmitter 142 in the control module. In the transducer, the first wireless receiver 144 delivers the signal to a first digital decoder 152, which decodes the signal and delivers the resulting analog information to a demodulator 170. The demodulator 170 demodulates the analog information and delivers the waveform to the treatment surface 127 of the transducer.

Each transducer may also comprise at least one sensor 154 for sensing the temperature of the patient's skin during treatment or for sensing a loss of contact between the transducer and the patient's skin. The sensor 154 communicates with a transducer switch 156, and the transducer switch 156 is configured to cease delivery of waveform information from the first digital decoder 152 to the treatment surface. The transducer may further comprise means attached to the surface of the transducer to alert the user to a malfunction. For example, the transducer may include at least one light emitting diode (LED) 158 on the visible surface of the transducer which lights up or flashes when a malfunction occurs. Alternatively, the transducer may include an audible alarm that alerts the user to the malfunction. The transducer may also include both an audible and visible alarm, such as a flashing LED coupled with an audible alarm.

The transducer switch 156 also communicates with a second digital encoder 157. The transducer switch 156 receives information regarding transducer use and malfunction and delivers the information to the second digital encoder 157. The second digital encoder 157 then digitally encodes the information and delivers it to a second wireless transmitter 150 in the transducer, which delivers the digitally encoded signal to a second wireless receiver 148 in the control module 113.

Like the control module 13 of device 10, the control module 113 of device 110 can also include a timer 128 and an alarm 132 in communication with a control switch 122. The timer 128, alarm 132 and control switch 122 operate in the same fashion as the timer 28, alarm 32 and switch 22 of the device 10. In this embodiment, however, the control module 113 further comprises a second wireless receiver 148 for receiving radio communication from the second wireless transmitter 150 in each of the transducers 126 or arrays 126a. The second wireless receiver 148 delivers the information received from the second wireless transmitter 150 in each of the transducers to a second digital decoder 160 which decodes the digital signal received from the second wireless transmitter 150 and delivers the decoded information to the control switch 122. The control switch 122 communicates with the first digital encoder 140 and is adapted to cease delivery of waveform information from the digital encoder 140 to the first wireless transmitter 142 based on the information received from the second digital encoder 160.

The control module 113 may further include at least one display screen 117 and an internal log 121 for documenting usage of the device 110 and treatment specifics. The at least one display screen 117 and internal log 121 operate in the same fashion as the at least one display screen 17 and internal log 21 of the control module 13.

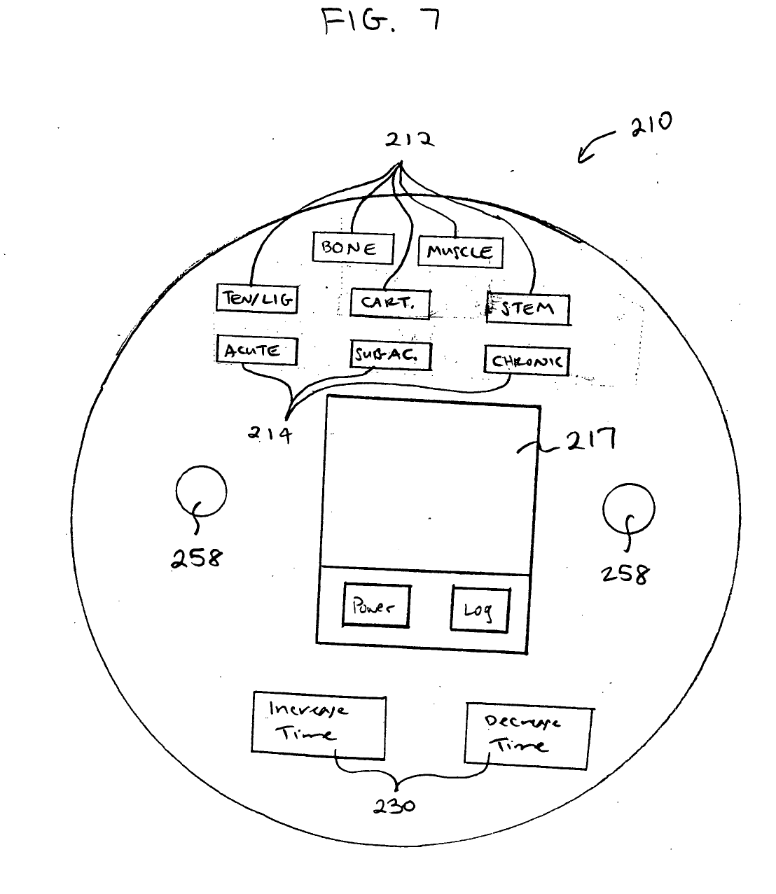

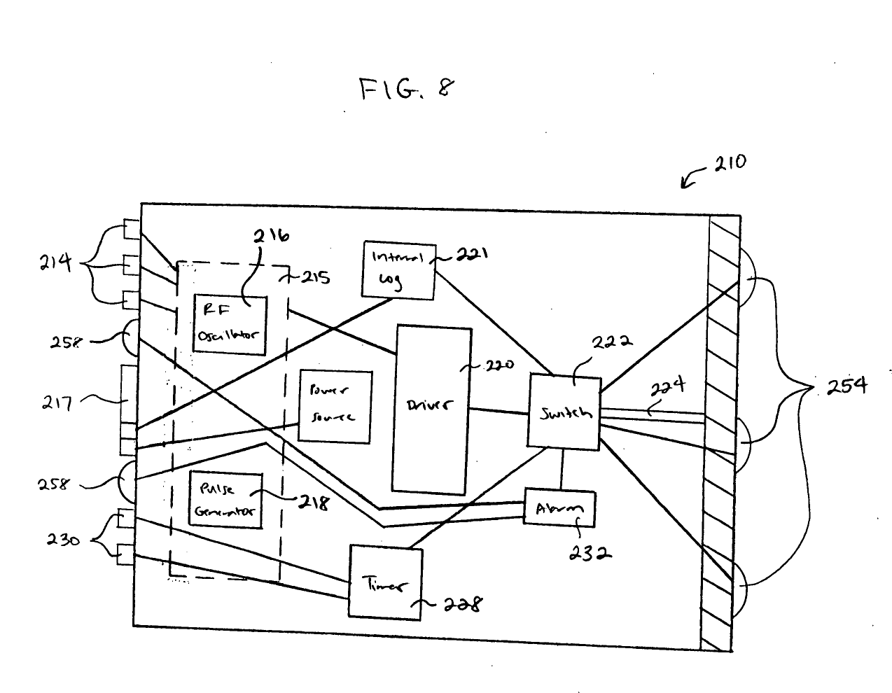

In yet another embodiment, as shown in FIGS. 7 and 8, the control module and transducer comprise a self-contained device 210 adapted to generate and transmit a waveform for a selected tissue type. The self-contained transducer device 210 is powered by an internal battery pack, rendering the device wireless and maximizing device portability.

In this embodiment, each self-contained transducer device 210 is designed to treat a single tissue type at a given time. Like the device 10, the transducer device 210 includes first selectors 212 for selecting tissue type, second selectors 214 for selecting injury type and third selectors 230 for setting the treatment time. The transducer device 210 varies the waveform based on the type of tissue and the type of injury selected.

In this embodiment, each of the first selectors 212, second selectors 214 and third selectors 230 communicate with a waveform generator 215, which comprises a RF oscillator 216 and a pulse generator 218. The RF oscillator 216 and pulse generator 218 operate in the same fashion as described above with respect to the RF oscillator 16 and pulse generator 18. In one embodiment, only one RF oscillator designed to deliver the appropriate frequency is provided. In another embodiment, the waveform generator includes a plurality of RF oscillators, one for each tissue type. However, because the device 210 is designed for maximum portability and to treat a single tissue type at a time, embodiments with only one RF oscillator may be more desirable.

In embodiments of the device 210 including a single RF oscillator 216, the RF oscillator may be programmed to deliver a frequency based on the tissue type selected with the first selector 212. Alternatively, the RF oscillator may be pre-programmed to deliver a single, specified frequency that is not user-selectable. In this embodiment, the need for the first selector 212 is eliminated.

Like the waveform generator 15, the waveform generator 215 communicates with a driver 220 which modulates the output of the RF oscillator 216 with the signal generated by the pulse generator 218. The driver 220 communicates with a switch 222, and the switch 222 communicates with the treatment surface. In use, the waveform generator 215 delivers a waveform to the driver 220, which delivers the waveform to the switch 222. The switch 222 then delivers the waveform to the treatment surface 226 via a cable 224, thereby initiating treatment.

Like the device 10, the device 210 can also include a timer 228 and an alarm 232 in communication with the switch 222. The timer 228, alarm 232 and switch 222 operate in the same fashion as the timer 28, alarm 32 and switch 22 of the device 10.

The device 210 may further include at least one display screen 217 and an internal log 221 for documenting usage of the device 210 and treatment specifics. The at least one display screen 217 and internal log 221 operate in the same fashion as the at least one display screen 17 and internal log 21 of the device 10.

The transducer device 210 may also comprise at least one sensor 254 for sensing the temperature of the patient's skin during treatment or for sensing a loss of contact between the transducer treatment surface 226 and the patient's skin. The temperature sensor 254 communicates with the switch 222, which is configured to cease delivery of waveform information to the transducer treatment surface 226 if the temperature of the patient's skin reaches a preprogrammed threshold level or if a loss of contact between the treatment surface and the patient's skin occurs. The transducer device 210 may further comprise means attached to the visible surface of the transducer device to alert the user to a malfunction. For example, the transducer device 210 may include at least one light emitting diode (LED) 258 on the visible surface of the transducer device 210 which lights up or flashes when a malfunction occurs. Instead of, or in addition to the LED, the transducer may include an audible alarm that alerts the user to the malfunction.

Because certain embodiments of the device 210 enable treatment of only a single tissue type, a kit comprising at least two devices 210, each designed to treat a different tissue type, may be provided. For example, one kit may include at least one device 210 designed to treat bone, and at least one device 210 designed to treat tendons or ligaments. Alternatively, a kit may include a plurality of devices 210 including at least one device for treating each type of tissue.

As noted above with respect to the second selectors 14a, 14b and 14c, more than one waveform may be used in each treatment session. If more than one waveform is used, the user may select how the device moves from one waveform to the next. Each waveform may be a distinct treatment point used for a selected period of time. Alternatively, the waveforms may be points on a sine wave and the device will gradually sweep from one waveform to the next in no less than 5 and no more than 10 steps. In this embodiment, the treatment time is divided generally equally between each step in the transition between waveforms. During the transition from one waveform to the next, only the pulse intensity will change until the next selected pulse intensity is achieved, at which time the remaining characteristics of the next waveform will be generated. This process continues until the treatment elapses.

The variant waveforms of the pulses and pulse series generated by the devices of the present invention enable the static treatment of injuries. Previously, treatment with ultrasound required constant movement of the treatment elements (i.e. transducers) in order to avoid thermal damage to the target and surrounding tissue. However, the devices of the present invention eliminate the need for constantly moving the treatment elements by varying the waveform and providing temperature sensors to monitor tissue temperature during treatment. The static treatment of injuries enabled by the devices of the present invention significantly increases the ease of use of the machine, thereby significantly reducing user error and thermal tissue damage.

During each therapeutic treatment session with the devices of the present invention, the ultrasonic treatment waveform includes multiple waveform segments, each segment having different characteristics. The total treatment time is divided between these segments as determined by the user. The user may alter the waveform or the treatment time of each segment with the first, second and third selectors described above. Alternatively, the waveform segments can be pre-programmed into the device by the manufacturer and the user can select the pre-programmed waveform.

The waveform of each treatment segment differs from the preceding or subsequent waveform segment in at least one waveform characteristic such as pulse intensity, pulse duration, pulse ratio or pulse repetition rate. Waveforms having segments that vary in only one characteristic are said to by uni-variant (see FIG. 4a), and waveforms having segments that vary in more than one characteristic are said to be multi-variant (see FIG. 4b). During treatment, the device cycles through the selected waveform segments to treat the selected tissue type and produces each waveform segment for the selected time until all waveforms have been used and the treatment time elapses. This inventive cycling of varying waveform segments enables the safe application of therapeutic ultrasound through the static placement of transducer treatment elements. The varying waveform modulates and controls the power density received by the target tissue throughout the treatment and maintains the power density within safe and therapeutic levels.

In use, the region of the body (either human or animal) to be treated is first shaved and a coupling gel is applied to the skin. The transducers are then placed over the coupling gel and the device 10 is used to transmit ultrasound energy to the treatment area. If the treatment area is located underneath a cast or the like, a window is cut in the cast and the coupling gel and transducer are applied to the area through the window. The first and second selectors 12 and 14 are used to select tissue type and injury type, and treatment is commenced. During treatment, the transducers are held in place by any suitable attachment means.

Many injuries have both acute and chronic components. As a result, treatment of such injuries may begin by treating the injured tissue for the acute component, and later treating the tissue for the chronic component. Alternatively, the tissue may be treated for the chronic injury first and the acute injury later. In another alternative, both the acute and the chronic components of the injury are treated by varying the pulse intensity, duration, ratio and repetition rate during each treatment session.

Tables 1 and 2 below list exemplary treatment parameters for each tissue type. The parameters listed in Tables 1 and 2 are for the treatment of acute injuries to the indicated tissue type with uni-variant waveform segments through which the treatment device cycles throughout the treatment time. Increases in the listed peak intensities of about 15%, and a pulse ratio of 1:3 can be used to treat sub-acute injuries to the indicated tissue type. In addition, increases in the listed peak intensities of about 25%, and a pulse ratio of 1:2 can be used to treat chronic injuries to the indicated tissue type.

TABLE 1
Treatment parameters for acute injuries - Uni-variant waveform segments

| | | | TENDON/ | | | | | |
|---|---|---|---|---|---|---|---|---|
| | BONE | | LIGAMENT | | JOINT | | MUSCLE | |
| FREQUENCY | 1.5 | MHz | 3 | MHz | 2.5 | MHz | 1 | MHz |
| PULSE RATIO | 1:4 | | 1:4 | | 1:4 | | 1:4 | |
| PULSE FREQUENCY | 1,000 | Hz | 1,000 | Hz | 1,000 | Hz | 1,000 | Hz |
| TOTAL TREATMENT TIME | 30 | min. | 35 | min. | 35 | min. | 30 | min. |
| PULSE DURATION | 200 | μsec | 200 | μsec | 200 | μsec | 200 | μsec |

TABLE 2
Pulse intensities for acute injuries - Uni-variant waveform segments

| | | TENDON/ | | |
|---|---|---|---|---|
| | BONE | LIGAMENT | JOINT | MUSCLE |
| PHASE 1 | begin at | begin at | begin at | begin at |
| | 30 mW/cm², | 30 mW/cm², | 50 mW/cm², | 100 mW/cm², |
| | increase to | increase to | increase to | increase to |
| | 50 mW/cm² and | 50 mW/cm² and | 300 mW/cm² and | 500 mW/cm² and |
| | decrease back to | decrease back to | decrease back to | decrease back to |
| | 30 mW/cm² over | 30 mW/cm² over | 50 mW/cm² over | 100 mW/cm² over |
| | 5 min. period | 5 min. period | 5 min. period | 5 min. period |
| PHASE 2 | begin at | begin at | begin at | begin at |
| | 30 mW/cm², | 50 mW/cm², | 30 mW/cm², | 30 mW/cm², |
| | increase to | increase to | increase to | increase to |
| | 100 mW/cm² and | 300 mW/cm² and | 100 mW/cm² and | 200 mW/cm² and |
| | decrease back to | decrease back to | decrease back to | decrease back to |
| | 30 mW/cm² over | 50 mW/cm² over | 30 mW/cm² over | 30 mW/cm² over |
| | 5 min. period | 5 min. period | 5 min. period | 5 min. period |
| PHASE 3 | begin at | begin at | begin at | begin at |
| | 30 mW/cm², | 30 mW/cm², | 50 mW/cm², | 50 mW/cm², |
| | increase to | increase to | increase to | increase to |
| | 50 mW/cm² and | 100 mW/cm² and | 200 mW/cm² and | 300 mW/cm² and |
| | decrease back to | decrease back to | decrease back to | decrease back to |
| | 30 mW/cm² over | 30 mW/cm² over | 50 mW/cm² over | 50 mW/cm² over |
| | 5 min. period | 5 min. period | 5 min. period | 5 min. period |
| PHASE 4 | begin at | begin at | begin at | begin at |
| | 30 mW/cm², | 50 mW/cm², | 30 mW/cm², | 100 mW/cm², |
| | increase to | increase to | increase to | increase to |
| | 100 mW/cm² and | 300 mW/cm² and | 100 mW/cm² and | 500 mW/cm² and |
| | decrease back to | decrease back to | decrease back to | decrease back to |
| | 30 mW/cm² over | 50 mW/cm² over | 30 mW/cm² over | 100 mW/cm² |
| | 5 min. period | 5 min. period | 5 min. period | over 5 min. |
| | | | | period |
| PHASE 5 | begin at | begin at | begin at | begin at |
| | 30 mW/cm², | 30 mW/cm², | 50 mW/cm², | 30 mW/cm², |
| | increase to | increase to | increase to | increase to |
| | 50 mW/cm² and | 50 mW/cm² and | 300 mW/cm² and | 200 mW/cm² and |
| | decrease back to | decrease back to | decrease back to | decrease back to |
| | 30 mW/cm² over | 30 mW/cm² over | 50 mW/cm² over | 30 mW/cm² over |
| | 5 min. period | 5 min. period | 5 min. period | 5 min. period |
| PHASE 6 | begin at | begin at | begin at | begin at |
| | 30 mW/cm², | 50 mW/cm², | 30 mW/cm², | 50 mW/cm², |
| | increase to | increase to | increase to | increase to |
| | 100 mW/cm² and | 300 mW/cm² and | 100 mW/cm² and | 300 mW/cm² and |
| | decrease back to | decrease back to | decrease back to | decrease back to |
| | 30 mW/cm² over | 50 mW/cm² over | 30 mW/cm² over | 50 mW/cm² over |
| | 5 min. period | 5 min. period | 5 min. period | 5 min. period |
| PHASE 7 | — | begin at | begin at | — |
| | | 30 mW/cm², | 50 mW/cm², | |
| | | increase to | increase to | |
| | | 100 mW/cm² and | 200 mW/cm² and | |
| | | decrease back to | decrease back to | 0 |
| | | 30 mW/cm² over | 50 mW/cm² over | |
| | | 5 min. period | 5 min. period | |
| TOTAL | 30 min. | 35 min. | 35 min. | 30 min. |
| TREATMENT | | | | |
| TIME | | | | |

In the non-limiting examples of uni-variant waveforms listed in Tables 1 and 2, the pulse intensity is variable and begins at an initial setting (e.g. 30 mW/cm²) and increases to a peak intensity (e.g. 50 mW/cm²) in increments of no less than about 5 and no more than about 10 intensity points per treatment interval. Furthermore, as indicated by Table 2, treatment of injuries occurs in phases, each of which phases may have different initial and peak intensities. As shown in Table 2, each phase is about 5 minutes in length, in which time the intensity increases from the initial intensity to the peak intensity and decreases back to the initial intensity. The treatment time may vary depending on the type of tissue being treated. In addition, although not exemplified in Tables 1 and 2, the treatment time may vary depending on the type of injury sustained by the indicated tissue as well as the chronicity of the injury being treated. Although not illustrated in the Tables, it is understood that all parameters of the waveform are user-selectable and variable. These variable waveform characteristics include pulse intensity, pulse duration, pulse ratio and pulse repetition rate. This variability in waveform characteristics enables the static application of ultrasonic therapy.

Tables 3 through 6 below list other exemplary treatment parameters for each tissue type. Table 3 lists exemplary parameters for the treatment of bone with multi-variant waveform segments for a total treatment time of 45 minutes. Table 4 lists exemplary parameters for the treatment of tendons and ligaments with multi-variant waveform segments for a total treatment time of 40 minutes. Table 5 lists exemplary parameter for the treatment of joints with multi-variant waveform segments for a total treatment time of 40 minutes. Table 6 lists exemplary parameters for the treatment of muscle with multi-variant waveform segments for a total treatment time of 40 minutes. Like those of Tables 1 and 2, the parameters listed in Tables 3 through 6 are for the treatment of acute injuries to the indicated tissue type with multi-variant waveform segments through which the treatment device cycles throughout the treatment time. As with the uni-variant waveforms listed in Tables 1 and 2, increases in the listed peak intensities of about 15%, and a pulse ratio of 1:3 can be used to treat sub-acute injuries to the indicated tissue type. In addition, increases in the listed peak intensities of about 25%, and a pulse ratio of 1:2 can be used to treat chronic injuries to the indicated tissue type.

TABLE 3
Treatment parameters for acute injuries to bone - Multi-variant waveform segments

| | PHASE 1 | | PHASE 2 | | PHASE 3 | | PHASE 4 | | PHASE 5 | | PHASE 6 | |
|---|---|---|---|---|---|---|---|---|---|---|---|---|
| FREQUENCY | 1.5 | MHz | 1.5 | MHz | 1.5 | MHz | 1.5 | MHz | 1.5 | MHz | 1.5 | MHz |
| PULSE | 1:4 | | 1:2 | | 1:3 | | 1:4 | | 1:1 | | 1:4 | |
| RATIO | | | | | | | | | | | | |
| PULSE | 1,000 | Hz | 835 | Hz | 2,500 | Hz | 1,000 | Hz | 5,000 | Hz | 1,000 | Hz |
| FREQUENCY | | | | | | | | | | | | |
| PULSE | 200 | μsec | 400 | μsec | 100 | μsec | 200 | μsec | 100 | μsec | 200 | μsec |
| DURATION | | | | | | | | | | | | |
| PULSE | begin at | | begin at 50 | | begin at | | begin at | | begin at | | begin at 30 | |
| INTENSITY | 30 | | mW/cm² | | 30 | | 30 | | 30 | | mW/cm² | |
| | mW/cm² | | and | | mW/cm² | | mW/cm² | | mW/cm² | | and | |
| | and | | increase to | | and | | and | | and | | increase to | |
| | increase | | 100 | | increase to | | increase to | | increase to | | 50 | |
| | to 50 | | mW/cm² | | 200 | | 80 | | 100 | | mW/cm² | |
| | mW/cm² | | and | | mW/cm² | | mW/cm² | | mW/cm² | | and | |
| | and | | decrease | | and | | and | | and | | decrease | |
| | decrease | | back to | | decrease | | decrease | | decrease | | back to 30 | |
| | back to | | 50 mW/cm² | | back to 30 | | back to 30 | | back to 30 | | mW/cm² | |
| | 30 | | | | mW/cm² | | mW/cm² | | mW/cm² | | | |
| | mW/cm² | | | | | | | | | | | |
| SEGMENT | 10 | min | 5 | min | 5 | min | 10 | min | 5 | min | 10 | min |
| TREATMENT | | | | | | | | | | | | |
| TIME | | | | | | | | | | | | |

TABLE 4
Treatment parameters for acute injuries to tendons
and ligaments - Multi-variant waveform segments

| | PHASE 1 | | PHASE 2 | | PHASE 3 | | PHASE 4 | | PHASE 5 | | PHASE 6 | |
|---|---|---|---|---|---|---|---|---|---|---|---|---|
| FREQUENCY | 3 | MHz | 3 | MHz | 3 | MHz | 3 | MHz | 3 | MHz | 3 | MHz |
| PULSE | 1:4 | | 1:2 | | 1:4 | | 1:3 | | 1:4 | | 1:1 | |
| RATIO | | | | | | | | | | | | |
| PULSE | 1,000 | Hz | 835 | Hz | 1,000 | Hz | 2,500 | Hz | 1,000 | Hz | 5,000 | Hz |
| FREQUENCY | | | | | | | | | | | | |
| PULSE | 200 | μsec | 400 | μsec | 200 | μsec | 100 | μsec | 200 | μsec | 100 | μsec |
| DURATION | | | | | | | | | | | | |
| PULSE | begin at | | begin at 50 | | begin at | | begin at | | begin at | | begin at 30 | |
| INTENSITY | 30 | | mW/cm² | | 30 | | 30 | | 30 | | mW/cm² | |
| | mW/cm² | | and | | mW/cm² | | mW/cm² | | mW/cm² | | and | |
| | and | | increase to | | and | | and | | and | | increase to | |
| | increase | | 300 | | increase to | | increase to | | increase to | | 100 | |
| | to 50 | | mW/cm² | | 80 | | 200 | | 50 | | mW/cm² | |
| | mW/cm² | | and | | mW/cm² | | mW/cm² | | mW/cm² | | and | |
| | and | | decrease | | and | | and | | and | | decrease | |
| | decrease | | back to 50 | | decrease | | decrease | | decrease | | back to 30 | |
| | back to | | mW/cm² | | back to 30 | | back to 30 | | back to 30 | | mW/cm² | |
| | 30 | | | | mW/cm² | | mW/cm² | | mW/cm² | | | |
| | mW/cm² | | | | | | | | | | | |
| SEGMENT | 10 | min | 5 | min | 5 | min | 5 | min | 10 | min | 5 | min |
| TREATMENT | | | | | | | | | | | | |
| TIME | | | | | | | | | | | | |

TABLE 5
Treatment parameters for acute injuries to joints - Multi-variant waveform segments

| | PHASE 1 | | PHASE 2 | | PHASE 3 | | PHASE 4 | | PHASE 5 | | PHASE 6 | |
|---|---|---|---|---|---|---|---|---|---|---|---|---|
| FREQUENCY | 2.5 | MHz | 2.5 | MHz | 2.5 | MHz | 2.5 | MHz | 2.5 | MHz | 2.5 | MHz |
| PULSE | 1:4 | | 1:4 | | 1:2 | | 1:3 | | 1:4 | | 1:1 | |
| RATIO | | | | | | | | | | | | |
| PULSE | 2,000 | Hz | 1,000 | Hz | 835 | Hz | 1,250 | Hz | 1,000 | Hz | 5,000 | Hz |
| FREQUENCY | | | | | | | | | | | | |
| PULSE | 100 | μsec | 200 | μsec | 400 | μsec | 200 | μsec | 200 | μsec | 100 | μsec |
| DURATION | | | | | | | | | | | | |
| PULSE | begin at | | begin at 30 | | begin at | | begin at | | begin at | | begin at 50 | |
| INTENSITY | 50 | | mW/cm² | | 50 | | 30 | | 30 | | mW/cm² | |
| | mW/cm² | | and | | mW/cm² | | mW/cm² | | mW/cm² | | and | |
| | and | | increase to | | and | | and | | and | | increase to | |
| | increase | | 80 | | increase to | | increase to | | increase to | | 100 | |
| | to 300 | | mW/cm² | | 200 | | 100 | | 50 | | mW/cm² | |
| | mW/cm² | | and | | mW/cm² | | mW/cm² | | mW/cm² | | and | |
| | | | decrease | | and | | and | | | | decrease | |
| | | | back to 30 | | decrease | | decrease | | | | back to 30 | |
| | | | mW/cm² | | back to 50 | | back to 30 | | | | mW/cm² | |
| | | | | | mW/cm² | | mW/cm² | | | | | |
| SEGMENT | 5 | min | 10 | min | 5 | min | 5 | min | 10 | min | 5 | min |
| TREATMENT | | | | | | | | | | | | |
| TIME | | | | | | | | | | | | |

TABLE 6
Treatment parameters for acute injuries to muscle - Multi-variant waveform segments

| | PHASE 1 | | PHASE 2 | | PHASE 3 | | PHASE 4 | | PHASE 5 | | PHASE 6 | |
|---|---|---|---|---|---|---|---|---|---|---|---|---|
| FREQUENCY | 1 | MHz | 1 | MHz | 1 | MHz | 1 | MHz | 1 | MHz | 1 | MHz |
| PULSE | 1:1 | | 1:4 | | 1:2 | | 1:4 | | 1:1 | | 1:4 | |
| RATIO | | | | | | | | | | | | |
| PULSE | 1,000 | Hz | 1,000 | Hz | 835 | Hz | 2,000 | Hz | 1,665 | Hz | 1,000 | Hz |
| FREQUENCY | | | | | | | | | | | | |
| PULSE | 500 | μsec | 200 | μsec | 400 | μsec | 100 | μsec | 300 | μsec | 200 | μsec |
| DURATION | | | | | | | | | | | | |
| PULSE | begin at | | begin at 30 | | begin at | | begin at | | begin at | | begin at 30 | |
| INTENSITY | 500 | | mW/cm² | | 300 | | 30 | | 400 | | mW/cm² | |
| | mW/cm² | | and | | mW/cm² | | mW/cm² | | mW/cm² | | and | |
| | and | | increase to | | and | | and | | and | | increase to | |
| | increase | | 100 | | increase to | | increase to | | increase to | | 80 | |
| | to 1.5 | | mW/cm² | | 1 W/cm² | | 200 | | 1.2 W/cm² | | mW/cm² | |
| | W/cm² | | and | | | | mW/cm² | | and | | and | |
| | | | decrease | | | | and | | decrease | | decrease | |
| | | | back to 30 | | | | decrease | | back to | | back to 30 | |
| | | | mW/cm² | | | | back to 30 | | 400 | | mW/cm² | |
| | | | | | | | mW/cm² | | mW/cm² | | | |
| SEGMENT | 3 | min | 10 | min | 5 | min | 7 | min | 5 | min | 10 | min |
| TREATMENT | | | | | | | | | | | | |
| TIME | | | | | | | | | | | | |

Although principally described for treating injuries to certain tissues, the devices of the present invention can be used for any suitable purpose. For example, the devices can be used to treat joints and joint capsules. In addition, the devices can be used to promote the differentiation and/or maturation of stem cells within a human or animal body.

In another embodiment of the present invention, stem cell therapy is used in conjunction with therapeutic ultrasound treatment. In this embodiment, stem cells are first harvested from bone marrow, adipose or other tissue or peripheral blood, or from an embryo or umbilical cord. The harvested stem cells are then implanted into the injured target tissue by injecting them intralesionally, intravenously, intrathecally, intraarticularly or the like. After injection of the stem cells, the treatment area is treated with ultrasound therapy. Upon treatment of the injected tissue with ultrasound therapy, the injured tissue, the stem cells and the ultrasound synergistically promote tissue healing, growth, regeneration and repair.

According to this exemplary embodiment, the stem cells can be injected into the tissue before, after or concurrently with the ultrasound treatment, and the ultrasound treatment would utilize pulsed or continuous waveforms having frequencies ranging from about 50 kHZ to about 3 MHz and intensities ranging from about 20 mW to about 3 W. These methods of treatment (i.e. stem cell therapy used in conjunction with ultrasound treatment) can be used to treat several tissue types, including but not limited to bone, muscle, tendons and/or ligaments and cartilage.

In yet another embodiment of the present invention, bioactive compounds are used in conjunction with ultrasound therapy and stem cell therapy. According to this embodiment at least one of the stem cells, target tissue and body surrounding the target tissue is treated with a bioactive compound. The bioactive compounds assist the differentiation and/or maturation of the stem cells, and are added to the stem cells, tissue or body either before or after the stem cells are injected in the target tissue and before, after or during the ultrasound therapy. Non-limiting examples of suitable bioactive compounds include dexamethasone, TGF-beta, IGF-1, BMP-2, CDMP-2, FGF-1, all members of the bone morphogenic protein family including all cartilage-derived morphogenic proteins, all members of the tissue and transforming growth factor families, all member of the insulin-like growth factor family, all members of the fibroblast growth factor family, hyaluronans and their derivatives, and any other growth factors appropriate for assisting the differentiation and/or maturation of stem cells.

One method for treating injured tissue according to this embodiment includes first harvesting stem cells from bone marrow, adipose or other tissue or peripheral blood from an embryo, fetus, adult or an umbilical cord. The stem cells are treated with the bioactive compounds either in vitro or in vivo. The treatment area within the body may also be treated with the bioactive compound before or after stem cell injection, and before, after or concurrently with the ultrasound treatment. The stem cells are injected into the treatment area either before or after being treated with the bioactive compound. As noted above, the stem cells may be injected in any suitable manner, such as intralesionally, intravenously, intramuscularly, intrathecally, intraarticularly, etc. The treatment area is then treated with ultrasound. Upon treatment of the injected tissue with ultrasound therapy, the injured tissue, the stem cells, the bioactive compounds and the ultrasound synergistically promote tissue healing, growth, regeneration and repair. These methods of treatment (i.e. stem cell therapy used in conjunction with ultrasound treatment) can be used to treat several tissue types, including but not limited to bone, muscle, tendons and/or ligaments and cartilage.

The preceding description has been presented with reference to certain exemplary embodiments of the present invention. However, workers skilled in the art and technology to which this invention pertains will appreciate that alterations and changes to the described embodiments may be practiced without meaningfully departing from the principal, spirit and scope of this invention. Accordingly, the foregoing description should not be read as pertaining only to the precise embodiments described and illustrated in the accompanying drawings, but rather should be read consistent with and as support for the following claims which are to have their fullest and fairest scope.

What is claimed is:

1. An ultrasound therapy device comprising:
a control module;
at least one transducer in communication with a driver housed in the control module;
a waveform generator in communication with the driver, wherein the waveform generator is adapted to generate a waveform comprising a plurality of different waveform segments, wherein each waveform segment comprises at least one waveform characteristic different from at least one other waveform segment;
wherein the driver receives the waveform from the waveform generator and delivers the waveform to the at least one transducer.

2. An ultrasound therapy device according to claim 1, wherein the waveform characteristics are selected from the group consisting of pulse intensities, pulse durations, pulse ratios, pulse repetition rates, and combinations thereof.

3. An ultrasound therapy device according to claim 1, wherein the at least one transducer comprises a plurality of transducers.

4. An ultrasound therapy device according to claim 1, wherein the at least one transducer comprises at least one transducer array.

5. An ultrasound therapy device according to claim 1, further comprising at least one sensor in communication with the at least one transducer, wherein the sensor is selected from the group consisting of sensors for sensing temperature and sensors for sensing a loss of contact between the transducer and a treatment site.

6. An ultrasound therapy device according to claim 1, further comprising:
a switch in communication with the driver and the at least one transducer; and
a timer in communication with the switch, the timer being programmable to have a start time and a stop time;
wherein, at the start time, the switch delivers the signal from the driver to the at least one transducer, and at the stop time, the switch ceases delivery of the signal to the transducer.

7. An ultrasound therapy device according to claim 6, further comprising an alarm in communication with the switch, wherein at the stop time, the switch energizes the alarm.

8. An ultrasound therapy device according to claim 6, wherein the switch pauses the delivery of the signal to the transducer upon a loss of contact between the transducer and a treatment site.

9. An ultrasound therapy device according to claim 1, wherein the waveform generator generates a waveform having a frequency ranging from about 50 kHz to about 3 MHz.

10. An ultrasound therapy device according to claim 1, wherein the driver outputs a signal having an intensity ranging from about 20 mW/cm² to about 3 W/cm².

11. An ultrasound therapy device according to claim 1, wherein the driver outputs a signal having an intensity ranging from about 25 to about 1.5 W/cm².

12. An ultrasound therapy device according to claim 1, wherein the waveform generator generates a pulse having a pulse duration ranging from about 10 to about 2,500 μs, a pulse ratio ranging from about 1:1 to about 1:8, a pulse repetition rate ranging from about 50 to about 10,000 Hz and a pulse intensity ranging about 20 mW/cm² to about 3 W/cm².

13. An ultrasound therapy device according to claim 1, wherein the waveform generator generates a pulse having a repetition rate ranging from about 500 Hz to about 2,500 Hz and a pulse duration ranging from about 100 to about 500 μs.

14. An ultrasound therapy device according to claim 1, wherein the device cycles through the plurality of waveform segments until each waveform segments has been used and a desired treatment time has elapsed.

15. An ultrasound therapy device according to claim 1, wherein at least one waveform segment comprises a plurality of waveform characteristics different from at least one other waveform segment.

16. An ultrasound therapy device according to claim 1, further comprising an internal log, wherein the log stores data regarding use of the device.

17. An ultrasound therapy device according to claim 16, further comprising at least one display screen for displaying the information stored by the log.

18. An ultrasound therapy device according to claim 16, wherein the at least one display screen comprises first and second display screens, the first display screen adapted to display information stored by the log and the second display screen adapted to display time elapsed during treatment.

19. An ultrasound therapy device according to claim 1, wherein the waveform generator comprises:
a RF oscillator adapted to generate a plurality of different user-selectable frequencies; and
a pulse generator adapted to generate a plurality of pulses having different waveform characteristics.

20. An ultrasound therapy device according to claim 1, wherein the waveform generator comprises:
a plurality of RF oscillators, each RF oscillator adapted to generate a different frequency, wherein the frequency delivered to the driver is user-selectable; and
a pulse generator adapted to generate a plurality of pulses having different waveform characteristics.

21. An ultrasound therapy device according to claim 1, wherein the transducer communicates with the control module by a cable.

22. An ultrasound therapy device according to claim 1, wherein the transducer wirelessly communicates with the control module.

23. An ultrasound therapy device according to claim 22, wherein the transducer communicates with the control module by radio.

24. An ultrasound therapy device according to claim 1, wherein the control module and transducer comprise a single, self-contained device, the transducer comprising a transducer treatment surface on one surface of the self-contained device, the control module being located on another surface of the device.

25. A method of treating injured tissue in a body comprising:
identifying the area of the body containing the injured tissue;
identifying the type of injured tissue;
attaching at least one ultrasound transducer to the area of the body containing the injured tissue;
selecting a frequency for operation of the ultrasound transducer based on the type of injured tissue;
selecting waveform characteristics for pulses generated by the ultrasound transducer based on the type of injury; and
activating the ultrasound transducer to treat the injured tissue with an ultrasound waveform having the selected frequency and the selected waveform characteristics, wherein the waveform comprises a plurality of waveform segments wherein at least one waveform segment has at least one different waveform characteristic than another waveform segment.

26. A method according to claim 25, wherein the waveform characteristics are selected from the group consisting of pulse intensities, pulse durations, pulse ratios, pulse repetition rates and combinations thereof.

27. A method according to claim 25, wherein the tissue is selected from the group consisting of bone, muscle, cartilage, tendons, ligaments and stem cells.

28. A method according to claim 25, wherein the injured tissue is treated in at least one session per day, the session ranging from about 5 to about 60 minutes.

29. A method according to claim 25, wherein the injured tissue is treated in from one to three sessions per day, each session ranging from about 5 to about 60 minutes.

30. A method according to claim 28, wherein each session ranges from about 20 to about 45 minutes.

31. A method according to claim 25, further comprising:
harvesting stem cells from a source; and
implanting the harvested stem cells in the injured tissue.

32. A method according to claim 31, wherein the stem cells are harvested from a source selected from the group consisting of tissue from an embryo, peripheral blood from an embryo, tissue from an umbilical cord, peripheral blood from an umbilical cord, embryonic bone marrow, fetal bone marrow, adult bone marrow, adult peripheral blood and adult adipose tissue.

33. A method according to claim 31, further comprising treating the stem cells with a bioactive compound.

34. A method according to claim 31, further comprising treating the injured tissue with a bioactive compound.

35. A method according to claim 33, further comprising treating the injured tissue with a bioactive compound.

36. A method according to claim 33, wherein the bioactive compounds is selected from the group consisting of TGF-beta, IGF-1, BMP-2, CDMP-2, FGF-1, bone morphogenic proteins, cartilage-derived morphogenic proteins, tissue growth factors, transforming growth factors, insulin-like growth factors, fibroblast growth factors and hyaluronans.

37. A method according to claim 34, wherein the bioactive compounds is selected from the group consisting of TGF-beta, IGF-1, BMP-2, CDMP-2, FGF-1, bone morphogenic proteins, cartilage-derived morphogenic proteins, tissue growth factors, transforming growth factors, insulin-like growth factors, fibroblast growth factors and hyaluronans.

38. A method of treating injured tissue in a body comprising:
identifying the area of the body containing the injured tissue;
identifying the type of injured tissue;
harvesting stem cells from a source;
implanting the harvested stem cells in the injured tissue;
attaching at least one ultrasound transducer to the area of the body containing the injured tissue; and
activating the ultrasound transducer to treat the injured tissue with an ultrasound waveform.

39. A method according to claim 38, wherein the stem cells are harvested from a source selected from the group consisting of tissue from an embryo, peripheral blood from an embryo, tissue from an umbilical cord, peripheral blood from an umbilical cord, embryonic bone marrow, fetal bone marrow, adult bone marrow, adult peripheral blood and adult adipose tissue.

40. A method according to claim 38, wherein the stem cells are implanted in the injured tissue before, after or during treatment of the tissue with the ultrasound waveform.

41. A method according to claim 38, further comprising treating the stem cells with a bioactive compound.

42. A method according to claim 38, further comprising treating the injured tissue with a bioactive compound.

43. A method according to claim 42, further comprising treating the stem cells with a bioactive compound.

44. A method according to claim 41, wherein the stem cells are treated with the bioactive compound before or after implantation of the stem cells in the injured tissue, and before, after or during treatment of the tissue with the ultrasound waveform.

45. A method according to claim 42, wherein the injured tissue is treated with the bioactive compound before or after implantation of the stem cells in the injured tissue, and before, after or during treatment of the tissue with the ultrasound waveform.

46. A method according to claim 38, wherein the ultrasound waveform comprises a plurality of waveform segments, wherein at least one waveform segment has at least one waveform characteristic different from another waveform segment.
